(19) United States
(12) Patent Application Publication
(10) Pub. No.: US 2017/0333225 A1
(43) Pub. Date: Nov. 23, 2017

(54) PROSTHESIS CARRING BARREL SUCTION-ATTACHING JOINT WITH LOCKING FUNCTION

(71) Applicant: KEN DALL ENTERPRISE CO., LTD., New Taipei City (TW)

(72) Inventors: Chia-Pao Cheng, New Taipei City (TW); Hung-Bin Chen, New Taipei City (TW); Hsiang-Ming Wu, New Taipei City (TW)

(21) Appl. No.: 15/157,416

(22) Filed: May 18, 2016

(51) Int. Cl. A61F 2/80; A61F 2/78

(52) U.S. Cl. CPC A61F 2/80; A61F 2002/805; A61F 2002/7818; A61F 2002/7868

(57) ABSTRACT

A prosthesis carrying barrel suction-attaching joint with locking function, particularly a joint specifically for suction-attachment to a carrying barrel, includes a positioning mechanism, which forms, in a center thereof, a positioning pin hole for receiving a positioning pin to dispose therein. The positioning mechanism is provided, in a lateral direction, with a penetration passage and a vacuum release passage in communication with the positioning pin hole. The penetration passage has two ends on opposite sides of the positioning pin hole. One of the ends is provided with a regulation assembly and another one provided with a locking assembly. The locking assembly includes a constraint block for retaining the positioning pin so that a chamber for vacuum regulation is formed in the site where the regulation assembly is arranged. Pressing down the locking assembly allows external air to flow through the vacuum release passage into the chamber to release the vacuum.

### TECHNICAL FIELD OF THE INVENTION

The present invention relates generally to a prosthesis carrying barrel suction-attaching joint with locking function, which is provided for regulating tightness between a stump of a user and a prosthesis and is characterized in that a vacuum condition of a carrying barrel can be released through one button actuation, while simultaneously releasing a locking pin of an internal lining sleeve of the prosthesis, so as to simplify a complicated process of dismounting the prosthesis.

### DESCRIPTION OF THE PRIOR ART

To mount a prosthesis on a user, a stump is often first covered with a prosthesis lining sleeve that is made of a soft material and is set to mate a carrying barrel of a corresponding size. An assisting device is operated to remove, through evacuation, excessive air between the stump and the prosthesis carrying barrel in order to maximize vacuum therebetween. However, heretofore, to prevent detachment of the prosthesis resulting from breaking or failure of air-sealing measures, the vacuum regulation structure is often used in combination with a locking structure. The two structures do not interfere with each other in the operations thereof, but for a user, complication and inconvenience of dismounting the prosthesis do indeed exit.

An example is U.S. Pat. No. 627,787, of which the feature is that a locking assembly is provide to mate, through a one-way ratchet, a locking pin. A knob is operated, through a spring, in combination with a centering shaft to cause the one-way ratchet to rotate or to be fixed, allowing the prosthesis lining sleeve locking pin to get into and out of the locking assembly. Although the locking assembly is also provided with a one-way relief air valve, no external connection with a vacuum evaluation device is provided, nor do a prosthesis carrying barrel suction force releasing mechanism. It can be easily appreciated that the locking assembly provides only a connection function, and is not provided for vacuum attachment of a prosthesis carrying barrel.

U.S. Pat. No. 6,334,876 features a locking mechanism arranged inside a carrying barrel and combined therewith to define an airtight space. When the locking mechanism is released, external air is simultaneously drawn in so that the suction force induced in the prosthesis carrying barrel is released. Such a structure is simple and shows an advantage in respect of reduced number of parts involved. However, such a device is not provided with a one-way air release valve and continuous release of air from the carrying barrel is not possible when an unlocking device is not kept in activation. Consequently, it is not possible to maintain the suction force of the carrying barrel during a period when a user walks. Further, this device is not provided with a measure of external connection with a vacuum evacuation device and is not provided for vacuum attachment of a prosthesis carrying barrel.

U.S. Pat. No. 8,211,187 features a slide valve comprising a channel that allows for evacuation to induce vacuum therein by external connection with a vacuum pump or introduction of external air therein to release the vacuum condition. Although a locking mechanism is provided in the carrying barrel, deactivation of the locking mechanism does not allow for simultaneous release of the suction force of the prosthesis carrying barrel. Consequently, with an attempt to take off the prosthesis, a user must first move off the slide valve and then pushes a unlocking device of the locking mechanism to enable release of connection and fixation of the prosthesis. In actual application, this device does help improve inconvenience of operation for the use, but the operability may be further improved.

WO 2015002955 A1 is another example and discloses a structure featuring an air passage formed in a locking pin, allowing for external connection with a vacuum pump to induce a vacuum condition inside the barrel. And, a locking device is provided to improve operation security. However, this device does not comprises a one-way air discharge valve and consequently, when the vacuum pump malfunctions, air that flows into the carrying barrel would break the vacuum condition. In addition, this device does not comprise a measure for releasing the vacuum condition. Apparently, there inconvenience of operability and further improvement may be needed.

In view of the forgoing, the prior art structures related to locking pins and vacuum regulation mechanism are complicated or they are constructed as separate designs in respect of functionalities thereof. It is desired to provide a solution that overcomes such drawbacks.

### SUMMARY OF THE INVENTION

In view of the above problem that the prior art structures are complicated and cannot be operated easily, an object of the present invention is to integrate or combine the functions or structures of vacuum regulation and coupling as one unitary device, this being the primary concern of the present invention.

To achieve the above object, the present invention provides a prosthesis carrying barrel suction-attaching joint with locking function, which comprises, structurally, a positioning pin that has a conical surface and is received in a positioning pin hole formed in a positioning mechanism. The positioning mechanism is provided, in a lateral direction, with a penetration passage and a vacuum release passage and in communication with the positioning pin hole. An end of the penetration passage is provided with a regulation assembly for regulating a vacuum condition and an opposite end is provided with a locking assembly that functions to fix or release the positioning pin.

By using the regulation assembly according to the present invention to conduct regulation of vacuum, and the locking assembly for easily releasing and fixing, the two functions of vacuum regulation and pin locking can be integrated together to allow a user to operate easily for timely adjustment for the best comfortableness.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view of the present invention.

FIG. 2 is an exploded view of the present invention.

FIG. 3 is a schematic view illustrating air flow when a locking assembly of the present invention is not actuated.

FIG. 3A is another schematic view illustrating the condition where the locking assembly of the present invention is not actuated.

FIG. 4 is a schematic view illustrating air flow when the locking assembly of the present invention is actuated.

FIG. 4A is another schematic view illustrating the condition where the locking assembly of the present invention is actuated.

FIG. 5 is a perspective view illustrating another embodiment of the present invention.

FIG. 6 is an exploded view of said another embodiment of the present invention.

FIG. 7 is a schematic view illustrating air flow when a locking assembly of said another embodiment of the present invention is not actuated.

FIG. 7A is another schematic view illustrating the condition where the locking assembly said another embodiment of the present invention is not actuated.

FIG. 8 is a schematic view illustrating air flow when the locking assembly of the present invention is actuated.

FIG. 8A is another schematic view illustrating the condition where the locking assembly of said another embodiment of the present invention is actuated.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

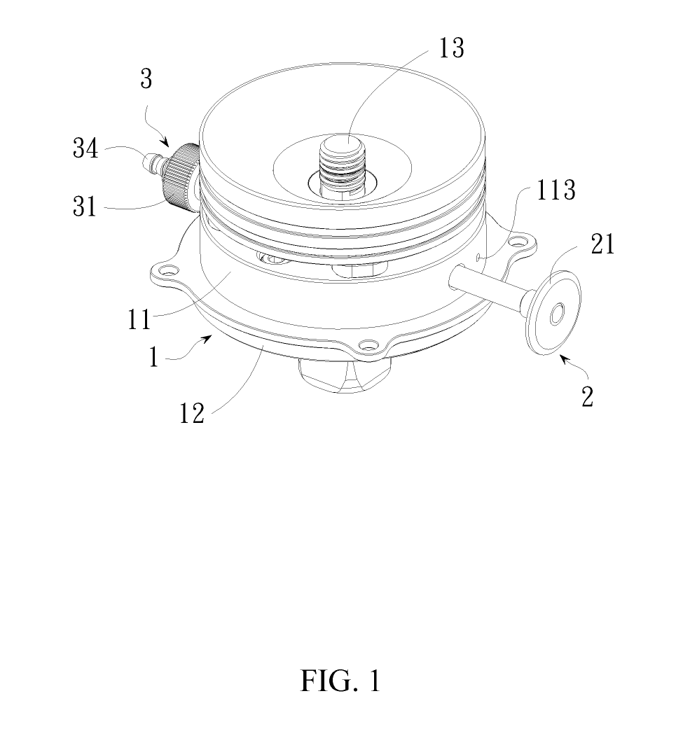

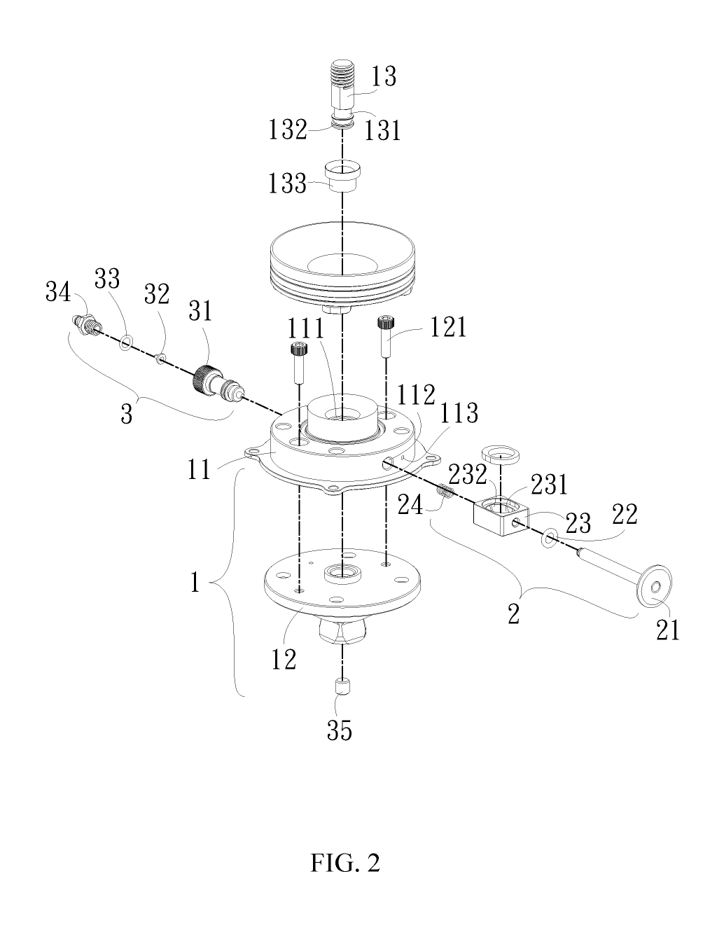

A preferred embodiment generally according to the present invention will be described, in detail, with reference to FIGS. 1 and 2 to help understand of the present invention. The present invention provides a prosthesis carrying barrel suction-attaching joint with locking function, and particularly a joint designed specification for suction-attachment of a carrying barrel lining sleeve (6), which comprises a structure that comprises a positioning mechanism (1). The positioning mechanism (1) generally comprises a base (11) and a support seat (12) that are fixed through screwing of a plurality of fixation screws (121), wherein the positioning mechanism (1) is provided, in a center thereof, with a positioning pin hole (111) for receiving a positioning pin (13) that extends through a positioning member (133) to be disposed therein. The positioning pin (13) is provided, in a portion below middle thereof, with a recessed groove (131), and the recessed groove (131) is connected to a conical surface (132). The positioning mechanism (1) is provided, in a lateral direction, with a penetration passage (112) and a vacuum release passage (113) in communication with the positioning pin hole (111). A regulation assembly (3) is arranged at an end of the penetration passage (112) and the regulation assembly (3) is made of a regulation valve pin (31), a one way valve (32), a seal ring (33), and a hose connector (34) that are coupled to each other in such a sequence. The one way valve (32) is provided, in a rightward portion thereof, with a first vacuum evacuation passage (115) in communication with a chamber (114) to be described later. The above-mentioned parts of the regulation assembly (3) are each provided, in a center thereof, with a hollow for passage of airflow. However, it is noted here that such an arrangement provides a one way air valves that constrains fluid to flow in just one direction to pass through the penetration passage (112). A locking assembly (2) is arranged at an opposite end of the penetration passage (112) and the locking assembly (2) is made up of a push knob (21), a seal ring (22), a constraint block (23), and a spring (24) that are coupled to each other in such a sequence. The constraint block (23) is provided, in a longitudinal direction, with a closure hole (231). The positioning mechanism (1) is provided, at a bottom end (117) thereof, with a plug (35).

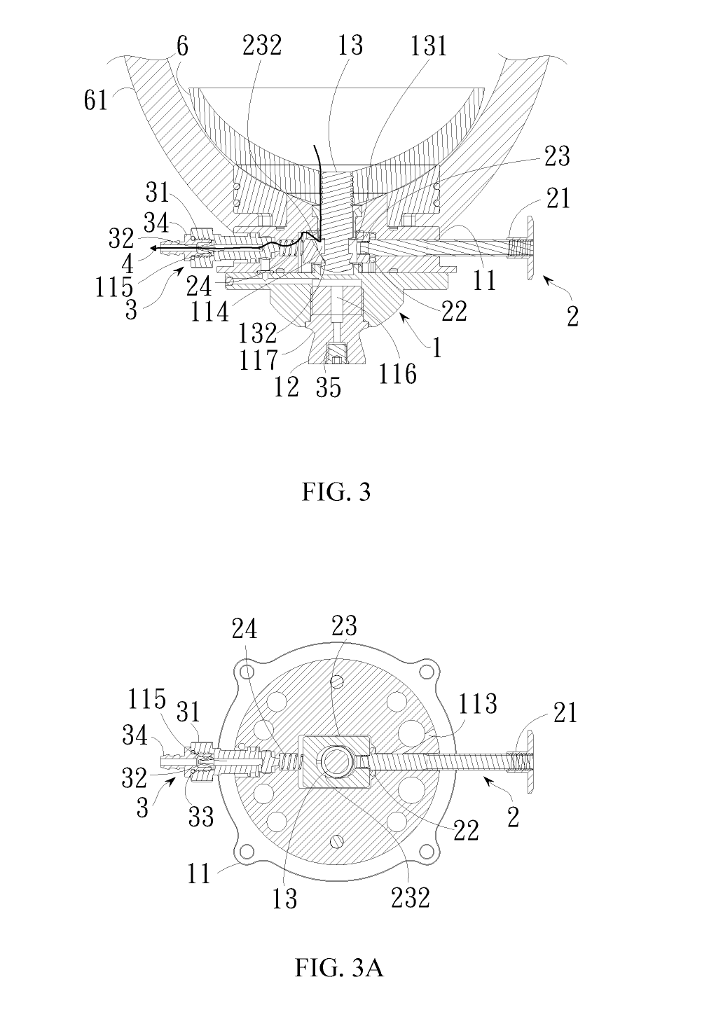

Referring to FIGS. 3-3A, the closure hole (231) is provided, on at least one half of an internal circumference thereof, with a contact surface (232) that is set in an inclined form. The inclination angle of the contact surface (232) corresponds, exactly, to the conical surface (132) of the positioning pin (13) such that the closure hole (231) may receive the positioning pin (13) to be fit, seated, and retained therein. The penetration passage (112) defines, at the end thereof where the regulation assembly (3) is provided, with respect to the recessed groove (131) of the positioning pin (13), a chamber (114), and the chamber (114) is in communication with the first vacuum evacuation passage (115) for connection with an external vacuum pump by means of the hose connector (34) of the regulation assembly (3) in order to evacuate air between the carrying barrel (61) and a carrying barrel lining sleeve (6) that are coupled thereto at an upper side and in communication with the chamber (114), such as an egress air flow A (4) for discharging as shown in the drawings, so that regulation of suction power is made possible. Further, a user may rotate or otherwise operate the regulation valve pin (31), as desired, in order to manually shut down the connection with the external vacuum pump for preventing excessive increase of suction power. The plug (35) functions to block the second vacuum evacuation passage (116) formed in the bottom (117) of the positioning mechanism (1) in order to prevent leakage of air from the inside that might causer over-operation of the vacuum pump.

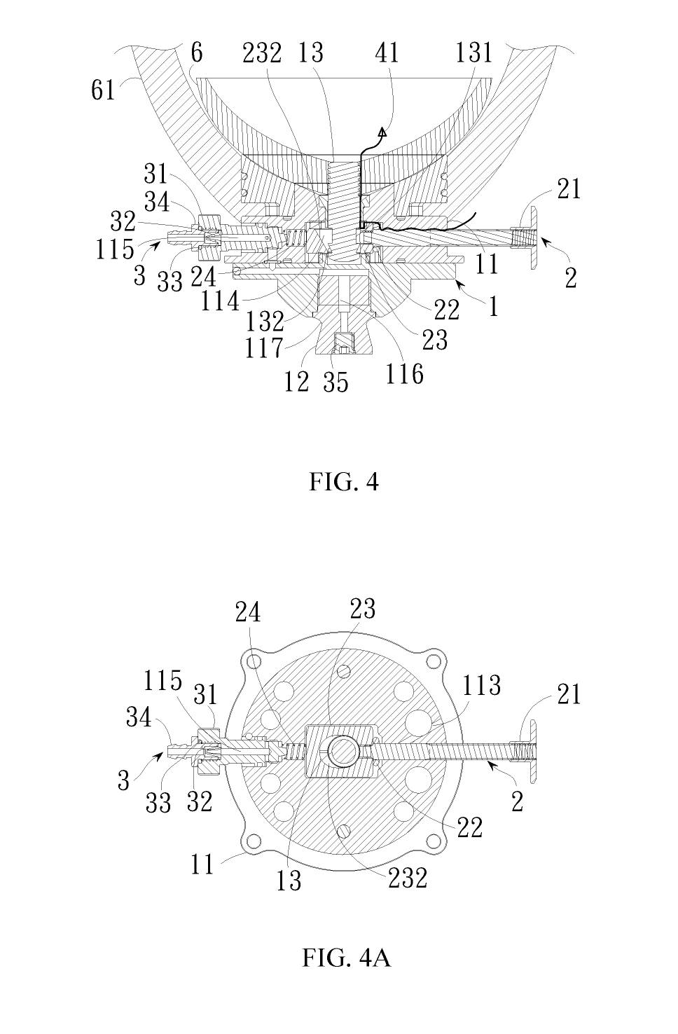

Referring to FIGS. 4-4A, with an attempt to dismount the prosthesis for rest, the user may push or depress, and thus operate, the locking assembly (2) such that the contact surface (232) is moved leftward and thus disengages and separates from the conical surface (132) of the positioning pin (13). Under this condition, an ingress air flow B (41) for introduction, as shown in the drawings, flows through the vacuum release passage (113) toward the carrying barrel lining sleeve (6) to thereby release a vacuum condition originally existing therein and allow the positioning pin (13) to be removed.

When the locking assembly (2) is not pushed or depressed, the spring (24) pushes and biases the constraint block (23) and the seal ring (22) is tightly interposed between the constraint block (23) and an inside flat plane of the base (11) to prevent air from passing through the penetration passage (112) to result in leakage of vacuum. When the locking assembly (2) is pushed or depressed, the seal ring (22) is moved together with the constraint block (23) so that the seal ring (22) is separated from the inside flat plane of the base (11) thereby breaking the sealing engagement and air is allowed to directly pass through the penetration passage (112) and the vacuum release passage (113) to flow into the chamber (114) and release the vacuum.

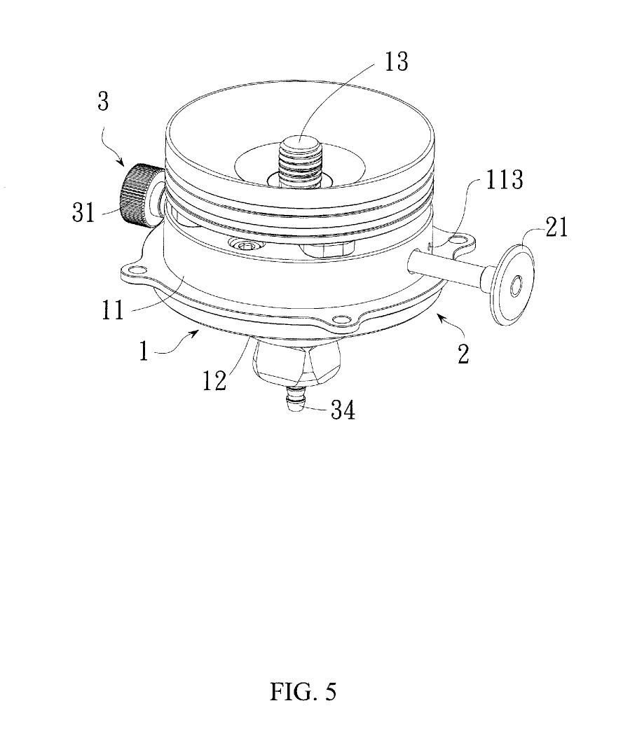

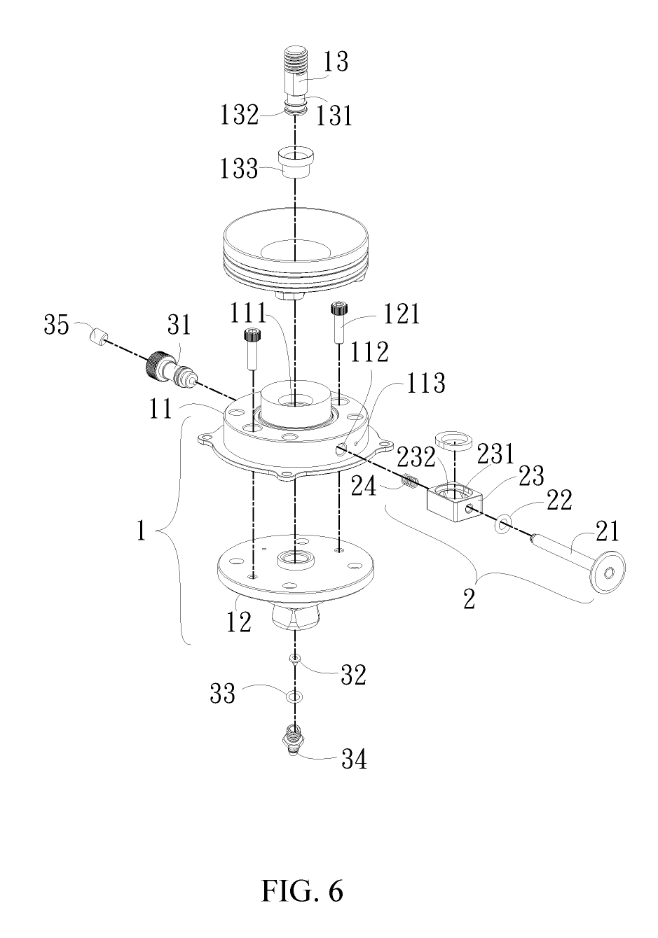

Another embodiment of the present invention, as shown in FIGS. 5-8A, comprises, in the structure thereof, a positioning mechanism (1). The positioning mechanism (1) generally comprises a base (11) and a support seat (12) that are fixed through screwing of a plurality of fixation screws (121), wherein the positioning mechanism (1) is provided, in a center thereof, with a positioning pin hole (111) for receiving a positioning pin (13) that extends through a positioning member (133) to be disposed therein. The positioning mechanism (1) is provided, at a bottom end (117) thereof, with a hose connector (34) that extends through a seal ring (33), to be fixed thereto. The positioning pin (13) is provided, in a portion below middle thereof, with a recessed groove (131), and the recessed groove (131) is connected to a conical surface (132). The positioning mechanism (1) is provided, in a lateral direction, with a penetration passage (112) and a vacuum release passage (113) in communication with the positioning pin hole (111). The penetration passage (112) is provided, at one end thereof, with a regulation valve pin (31) and a plug (35) that are coupled to each other in such a sequence to form a one way air valve that constrains fluid to flow in just one direction. Further, the penetration passage (112) is also provided with a second vacuum evacuation passage (116) extending to and in communication with the hose connector (34). A locking assembly (2) is arranged at an opposite end of the penetration passage (112) and the locking assembly (2) is made up of a push knob (21), a seal ring (22), a constraint block (23), and a spring (24) that are coupled to each other in such a sequence. The constraint block (23) is provided, in a longitudinal direction, with a closure hole (231).

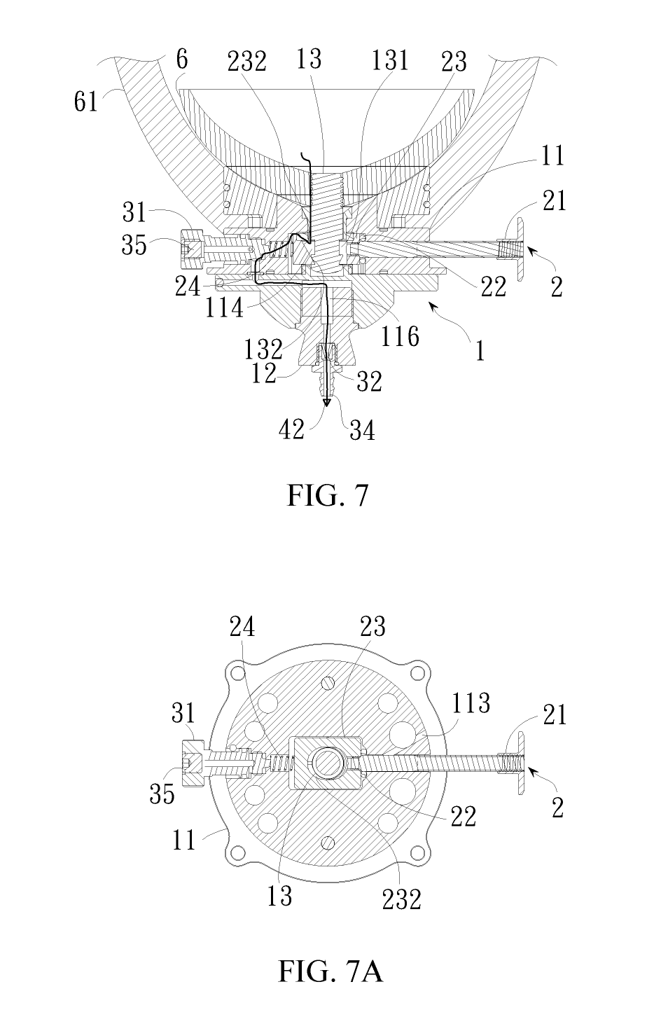

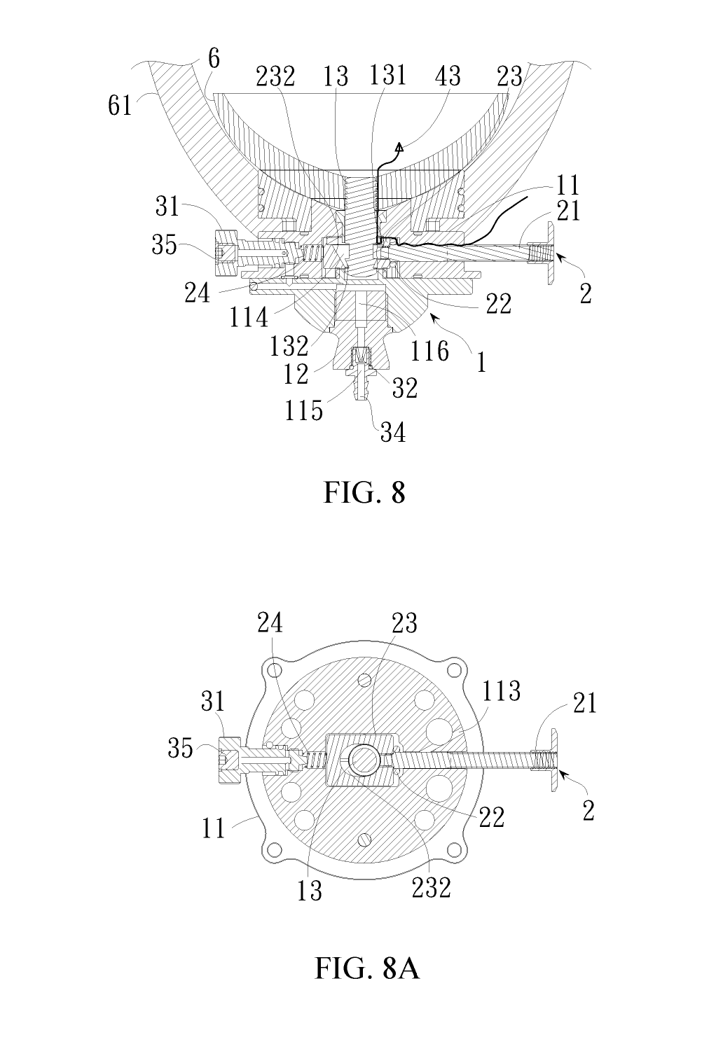

Referring to FIGS. 7-8A, similar to the previous embodiment, the closure hole (231) is provided with a contact surface (232) that is set in an inclined form and corresponds, exactly, to the conical surface (132) of the positioning pin (13). With the positioning pin (13) being fit, seated, and retained in the closure hole (231), the penetration passage (112) defines, at the end thereof where the regulation valve pin (31) is provided, with respect to the recessed groove (131) of the positioning pin (13), a chamber (114), the chamber (114) is used in combination with the second vacuum evacuation passage (116) that is in communication with the penetration passage (112) to connect with an external vacuum pump by means of the hose connector (34) in order to evacuate air between the carrying barrel (61) and a carrying barrel lining sleeve (6) that are coupled thereto at an upper side and in communication with the chamber (114), such as an egress air flow C (42) for discharging as shown in the drawings, so that regulation of suction power is made possible. When the best fitting condition of the user is reached, the externally connected vacuum pump can be manually shut down and the regulation valve pin (31) is rotated to block the connection between the chamber (114) and the second vacuum evacuation passage (116). When a user wishes to dismount the prosthesis for rest, the locking assembly (2) is pushed and depressed to have the contact surface (232) moved leftwards for disengaging and separating from the conical surface (132) of the positioning pin (13). Under this condition, external air is allowed to flow through the air aperture (113), as an ingress air flow D (43) illustrated, so as to flow from the vacuum release passage (113) toward the carrying barrel lining sleeve (6) to thereby release a vacuum condition originally existing therein and allow the positioning pin (13) to be removed.

When the locking assembly (2) is not pushed or depressed, the spring (24) pushes and biases the constraint block (23) and the seal ring (22) is tightly interposed between the constraint block (23) and an inside flat plane of the base (11) to prevent air from passing through the penetration passage (112) to result in leakage of vacuum. When the locking assembly (2) is pushed or depressed, the seal ring (22) is moved together with the constraint block (23) so that the seal ring (22) is separated from the inside flat plane of the base (11) thereby breaking the sealing engagement and air is allowed to directly pass through the penetration passage (112) and the vacuum release passage (113) to flow into the chamber (114) and release the vacuum.

The above description provided for two embodiments clearly illustrate, with reference to the drawings, that with the positions where the plug (35) and the hose connector (34) are arranged, the regulation structures of the two are different. The purpose of the present invention is to provide an integrated or combined arrangement for two functions of vacuum regulation and pin locking, making it easy for a user to operate and use and allowing for timely adjustment of comfortableness. Also, all connections involved are of standard specifications, allowing a user to interchange the joint or fitting without replacement of the carrying barrel, and may be used in combination with any vacuum product available in the market to provide the entire structure with excellent adaptability for assembly and use.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

1. A prosthesis carrying barrel suction-attaching joint with locking function, comprising:
a positioning pin, which is provided, in a portion below middle thereof, with a recessed groove, the recessed groove being connected to a conical surface; a positioning mechanism, which is provided, in a center thereof, with a positioning pin hole for receiving the positioning pin to dispose therein, the positioning mechanism being provided, in a lateral direction, with a penetration passage and a vacuum release passage in communication with the positioning pin hole; a regulation assembly, which is mounted to an end of the penetration passage and comprises a regulation valve pin that is rotatable for regulation vacuum and a hose connector coupled to the regulation valve pin; and a locking assembly, which is mounted to an opposite end of the penetration passage and comprises a push knob and a constraint block, the constraint block being provided therein with a closure hole;
wherein the closure hole receives the positioning pin to be fit and retained therein and the penetration passage defines, at a portion thereof where the regulation assembly is provided, a chamber, such that when the locking assembly is activated, the closure hole releases the fitting engagement with the positioning pin to allow air to enter through the vacuum release passage and thus breaking a vacuum condition of the chamber to thereby allow the positioning pin to be removed.

2. The prosthesis carrying barrel suction-attaching joint with locking function according to claim 1, wherein the locking assembly further comprises a spring, which is arranged at an end of the positioning block such that the spring abuts an internal side wall of the base; and a seal ring, which is arranged at an opposite end of the positioning block and is biased by the spring so that the seal ring is set in engagement with an internal side wall of the base, wherein through the activation of the locking assembly, the seal ring is separated from the internal side wall of the base to allow air to pass through a gap between the penetration passage and the locking assembly so as to release the vacuum condition.

3. The prosthesis carrying barrel suction-attaching joint with locking function according to claim 1, wherein the regulation assembly is structured as a one way air valve.

4. A prosthesis carrying barrel suction-attaching joint with locking function, comprising:
a positioning pin, which is provided, in a portion below middle thereof, with a recessed groove, the recessed groove being connected to a conical surface; a positioning mechanism, which is provided, in a center thereof, with a positioning pin hole for receiving the positioning pin to dispose therein, the positioning mechanism being provided, in a lateral direction, with a penetration passage and a vacuum release passage in communication with the positioning pin hole, the positioning mechanism being provided, at a bottom thereof, with a hose connector; a regulation valve pin, which is rotatable for regulation of vacuum and is mounted to an end of the penetration passage, the penetration passage comprising a second vacuum evacuation passage extending to and in communication with the hose connector; and a locking assembly, which is mounted to an opposite end of the penetration passage and comprises a push knob and a constraint block, the constraint block being provided therein with a closure hole;
wherein the closure hole receives the positioning pin to be fit and retained therein and the penetration passage defines, at a portion thereof where the regulation assembly is provided, a chamber that is operable with the hose connector to form vacuum, such that when the locking assembly is activated, the closure hole releases the fitting engagement with the positioning pin to allow air to enter through the vacuum release passage and thus breaking the vacuum condition of the chamber to thereby allow the positioning pin to be removed.

5. The prosthesis carrying barrel suction-attaching joint with locking function according to claim 4, wherein the regulation valve pin and the hose connector are each a one way air valve.

6. The prosthesis carrying barrel suction-attaching joint with locking function according to claim 4, wherein the locking assembly further comprises a spring, which is arranged at an end of the positioning block such that the spring abuts an internal side wall of the base; and a seal ring, which is arranged at an opposite end of the positioning block and is biased by the spring so that the seal ring is set in engagement with an internal side wall of the base, wherein through the activation of the locking assembly, the seal ring is separated from the internal side wall of the base to allow air to pass through a gap between the penetration passage and the locking assembly so as to release the vacuum condition.
